(19) United States
(12) Patent Application Publication
(10) Pub. No.: US 2010/0255050 A1
(43) Pub. Date: Oct. 7, 2010

(54) Polyethylene Based Bioactive Agents

(76) Inventors: James Klein Leonard, Gainesville, FL (US); Kenneth Boone Wagener, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/664,172

(22) PCT Filed: Jul. 24, 2008
(86) PCT No.: PCT/US2008/071020

§ 371 Date: Jun. 2, 2010

Related U.S. Application Data
(60) Provisional application No. 60/951,666, filed on Jul. 24, 2007.

(51) Int. Cl. A61F 2/00; A61K 9/70; A61P 43/00; A61K 31/765; C08F 36/02

(52) U.S. Cl. 424/422; 424/443; 424/78.18; 526/318.1

(57) ABSTRACT

The present invention concerns an acyclic diene metathesis (ADMET) chemistry-based method of making polymers incorporating biologically active molecules, and the polymers formed thereby. Functionalized polymers prepared by this method can be used to produce a broad range of commercially important products such as drag delivery agents (prodrugs), chromatography reagents (e.g., for use in separatory reagents), biomimetics, and biodegradable synthetic polymers.

### GOVERNMENT SUPPORT

The subject matter of this application has been supported by research grants from the National Science Foundation under grant numbers DMR-0703261 and DMR-0314110. Accordingly, the government has certain rights in this invention.

### BACKGROUND OF THE INVENTION

The term “metathesis” refers to a mutual transalkylidenation of alkenes and alkynes in the presence of catalysts. Reactions of this type are employed in many industrially important processes. A review may be found in: M. Schuster, S. Blechert, Angew. Chem., 1997, 109:2124; and S. Armstrong, J. Chem. Soc., Perkin Trans., 1998, 1:371. Metathesis reactions include the oligomerization and polymerization of acyclic dienes (ADMET) and the synthesis of carbocycles and heterocycles having various ring sizes by ring closing metathesis (RCM). Crossed metatheses of different alkenes are also known (Brummer, O. et al., Chem. Eur. J., 1997, 3:441). For the aforementioned metathesis reactions, it is possible to use, for example, the ruthenium-alkylidene compounds described in WO-A-93/20111, the ruthenium-based catalyst systems described by A. W. Stumpf, E. Saive, A. Deomceau and A. F. Noels in J. Chem. Soc., Chem. Commun., 1995, 1127-1128, or the catalyst systems described by P. Schwab, R. H. Grubbs and J. W. Ziller in J. Am. Chem. Soc., 1996, 118, 100 (see also WO 96/04289) as catalysts. The above publications, and all other publications, published patent applications, and patents cited hereafter, are incorporated herein by reference in their entirety.

Metathesis chemistry has received much attention as a method to obtain precise structural control in polymer synthesis. Recent advances include the synthesis of polyolefin and polyolefin-like polymers through two-step procedures involving ADMET polymerization or ring-opening metathesis polymerization (ROMP), followed by hydrogenation. Examples of such polymerizations include perfectly linear polyethylene (O'Gara, J. E., et al., die Makromomolekulare Chemie, 1993, 14:657; Grubbs, R. H. and W. Zhe, Macromolecules, 1994, 27:6700), telechelic polyethylene (Hillmyer, M. A., “The Preparation of Functionalized Polymers by Ring-Opening Metathesis Polymerization”, Ph.D. Dissertation, California Institute of Technology, 1995), ethylene/vinyl alcohol copolymers (Valenti, D. J. and K. B. Wagener, Macromolecules, 1998 31:2764) and polyethylene with precisely spaced alkyl side chains (Valenti, D. J. and K. B. Wagener, Macromolecules, 1997, 30:6688). Polymerized dienes and methods for preparing them by step propagation, condensation type polymerization of acyclic dienes are described in U.S. Pat. No. 5,110,885 (Wagener et al.) and U.S. Pat. No. 5,290,895 (Wagener et al.).

ADMET chemistry-based methods for making polymers incorporating amino acids or polypeptides, and the resulting polymers, are described in U.S. Pat. No. 6,680,051 (Wagener et al.) and U.S. Pat. No. 7,172,755 (Wagener et al.). FIG. 1 of U.S. Pat. No. 6,107,237 shows some examples of metathesis reactions that are useful for constructing molecules. In instances where it may be desirable that the products be free of carbon—carbon multiple bonds, conversion of these multiple bonds to single bonds (hydrogenation) can significantly influence physical and chemical properties, biological activity, oxidative stability, etc. Substrates may contain a wide group of functionalities. The potential scope of application of this methodology is vast. The overall result of this process is the formation of carbon—carbon single bonds. This is highly useful in organic synthesis. Unsaturated vegetable oils may be functionalized by cross-metathesis with functionalized olefins and then hydrogenated. Cyclic molecules may be constructed and then hydrogenated. Difunctional monomers with long aliphatic chains, which may otherwise be difficult to product, may easily be synthesized. U.S. Pat. No. 4,496,758 describes metathesis and cross-metathesis of alkenyl esters to produce unsaturated monomers which can be used in polymer synthesis. U.S. Pat. No. 5,146,017 describes metathesis of partially fluorinated alkenes.

Metathesis chemistry has been shown to be effective in the synthesis of a broad range of polymers. A common feature of all polymers produced via metathesis is unsaturation in the main chain. Oxidative stability can be increased by removal of this unsaturation. Therefore, polymers which may be difficult to synthesize (or even completely inaccessible) by other means may be produced by metathesis and then value added by saturating the double bonds. Other properties may be manipulated such as toughness, thermal stability, permeability, crystallinity, etc.

Metathesis polymers are often prepared, isolated, and purified prior to hydrogenation. Additional hydrogenating agents are then added and hydrogenation is effected. Disadvantages are loss of product during isolation and purification after the first step, the added effort to conduct reactions in additional vessels, use of additional reagents to effect hydrogenation, and the isolation and purification of the polymer from reagents used in the hydrogenation.

These syntheses typically involve first the synthesis and isolation of unsaturated polymers followed by a second hydrogenation step. Two of the more successful methods for hydrogenation are diimide reduction (Valenti, D. J. and K. B. Wagener, supra, 1997) and catalytic hydrogenation with Crabtree's iridium complex (Hillmyer, M. A., supra, 1995). The Valenti method requires an excess of the hydrogenating species and the Hillmyer method attains complete hydrogenation only if the olefin/catalyst ratio was kept less than or equal to 100:1.

McLain, et al. (McLain, S. J., et al., Proceedings PMSE, 1997, 76:246) reported a one-pot procedure for producing ethylene/methyl acrylate copolymers by the ROMP of ester-functionalized cycloolefins using Cl₂ (PCy₃)₂ Ru═CHCH═CPh₂ and then hydrogenating by simply applying hydrogen pressure to the completed ROMP reaction system. The metathesis catalyst residue was assumed to be converted to RuHCl(PCy₃)₂ in the presence of hydrogen gas. RuHCl(PCy₃)₂ is an effective hydrogenation catalyst. However, hydrogen pressures of at least 400 psi were required to maintain catalytic activity and achieve greater than 99% reduction.

U.S. Pat. No. 5,539,060 describes the one-pot ROMP of cyclic olefins and subsequent hydrogenation without the need for isolation of the polymer from the first step or deactivation of the olefin metathesis catalyst. However, metathesis is effected with a binary catalyst system (e.g., WCl₆/SnBut₄) and then another catalyst must be added for hydrogenation. Further, in some cases hydrogen halides can be produced in this process. An acid binder is required in these cases as such by-products can cause corrosion in reaction vessels.

There are advantages to systems in which olefin metathesis and the subsequent catalytic hydrogenation is conducted in a single vessel where the only added reagents are low cost support materials and hydrogen gas. It is often advantageous for quantitative hydrogenation to be achieved under mild conditions (e.g., low to moderate hydrogen pressures and temperatures) and for purification of the final product to be achieved by simple filtration and solvent removal (if used) with minimal loss of product. Such a method is described in U.S. Pat. No. 6,107,237 (Wagener et al.).

For some time, metathesis polymerization reactions and organic metathesis reactions forming small molecules required that a liquid state be achieved. In the case of metathesis polymerization reactions, the reaction proceeds via melt polymerization, often with the addition of other chemicals, such as solvents. Sometimes it is advantageous for metathesis to be performed at least in part in the solid state, allowing advantages associated with the use of low reaction temperatures (e.g., longer catalyst life) and solvent-less in-situ processing. U.S. Pat. No. 6,660,813 (Wagener et al.) describes an in-situ method for performing organic metathesis polymer chemistry in the solid state, which includes the step of providing an organic monomer and a catalyst, the catalyst for driving a metathesis polymerization reaction of the monomer. The organic monomer can be provided as a liquid monomer. The reaction produces reaction products including a polymeric end product and at least one volatile reaction product. At least a portion of the volatile reaction product is removed during the reaction to favor formation of the reaction product. The reaction can be performed at a temperature below the average melting point of the polymeric end product such that at least a portion of the reaction is performed in the solid phase. The reaction can comprise ADMET chemistry.

Polymer therapeutics refers to the use of polymers in biomedical applications and may involve biologically active polymers, polymer-drug conjugates, polymer-protein conjugates, and other covalent constructs of bioactive molecules (R. Duncan, “Polymer therapeutics for tumor specific delivery”, Chem. & Ind., 1997, 7:262-264). An exemplary class of a polymer-drug conjugate is derived from copolymers of hydroxypropyl methacrylamide (HPMA), which have been extensively studied for the conjugation of cytotoxic drugs for cancer chemotherapy (R. Duncan, Anti-Cancer Drugs, 1992, 5:210; D. Putnam et al., Adv. Polym. Sci., 1995, 122(55): 123; R. Duncan et al., STP Pharma, 1996, 6:23-263). The polymers used to develop polymer therapeutics may also be separately developed for other biomedical applications that require the polymer be used as a material. Thus, drug release matrices (including microparticles and nanoparticles), hydrogels (including injectable gels and viscous solutions) and hybrid systems (e.g., liposomes with conjugated poly(ethylene glycol) on the outer surface) and devices (including rods, pellets, capsules, films, gels) can be fabricated for tissue or site specific drug delivery. Polymers are also widely used clinically as excipients in drug formulation. Within these three broad application areas: (1) physiologically soluble molecules, (2) materials, and (3) excipients, biomedical polymers provide a broad technology platform for optimizing the efficacy of an active therapeutic agent.

Drugs have been reacted with an acrylate or other vinyl substituent, followed by purification of the vinyl drug monomers and polymerization via free radical polymerization. The resultant polymers have a tremendous drug-loading capability because every repeat unit has a drug molecule appended to it. A limitation of this approach is that it provides poor control over polymer architecture due to the multiple different side reactions that can be present from a radical polymerization. There can be no pre-determination of how much branching will be obtained with these polymers, and the polydispersity of these materials are often rather large. Solubility of these types of materials varies greatly with the solubility of the drug attached to the backbone along with the type of spacer utilized to connect the drug and the vinyl substituent.

It would be advantageous to have available a method that allows both the precise predetermination of polymer architecture and control of drug-loading, and the resulting polymers of such a production method.

### BRIEF SUMMARY OF THE INVENTION

The present invention concerns an acyclic diene metathesis (ADMET) chemistry-based method of making polymers incorporating biologically active (bioactive) molecules, and the polymers formed thereby. The invention provides a step-growth polymerization technique that allows both the precise predetermination of polymer architecture and control of drug-loading. The polydispersity of a step-growth polymerization was predicted to be 2, and the inventors are able to confirm this experimentally.

By using a diene acid precursor, such as 3,3 acid, 6,6 acid, 9,9 acid, or 18,18 acid monomers, the amount of biologically active molecule (drug) on the polymer backbone can be varied at exact intervals and thereby controlled. Having the capability to vary the drug-loading while simultaneously knowing the exact placement of the drug molecules on the polymer is a significant benefit for a drug delivery material.

Functionalized polymers prepared by the methods of the invention can be used to produce a broad range of commercially important products such as drug delivery agents (prodrugs), chromatography reagents (e.g., for use in separatory reagents), biomimetics, and biodegradable polymers. For example, branched functionalized polymers can be used as tissue culture substrates. Such polymers could also be used in an implantable medical device to modify the physiological response to the device.

Polymers of the invention can be used to make materials that biodegrade more quickly than conventional carbon-based linear polymers (e.g., polyethylene). Such materials can be fashioned into films for use in packaging, bags, and the like, that would quickly be degraded (e.g., by chemical or microorganism-mediated processes) in landfills. Similarly, such materials can be fashioned into medical implants designed to slowly degrade in vivo. For example, the material can be impregnated with a drug for sustained release. The material may also be fashioned into a scaffolding for applications in tissue engineering.

In one embodiment of the invention, the monomer is an alpha omega diene monomer with a biologically active molecule (such as a non-steroidal anti-inflammatory drug) covalently attached, which can be polymerized via a step growth condensation type polymerization to yield a polyethylene polymer with precise branches of the corresponding biologically active molecule. There are many examples of vinyl groups attached to a drug molecule in the scientific literature, but this is the first instance in which the precise location of the drug branches is known, and can be pre-determined by rational design.

Optionally, the polymers of the invention include one or more spacers connecting the biologically active molecule to the polymer backbone. The spacer utilized can vary from being hydrophobic to hydrophilic, and are preferably non-toxic.

The biologically active molecule can be cleaved from the polymer by chemical or enzymatic hydrolysis, yielding the polymer, which remains useful, e.g., as a coating, along with the biologically active molecule and the spacer (if present) can easily be eliminated from the body. Surface density of the biologically active molecule can be modulated with the varying number of methylene units between the terminal alkenes which directly translates to polymer architecture.

The polymers of the invention are useful in a wide variety of pharmaceutical and biomedical applications. For example, the polymers may be formulated as coatings for drug tablets, contact lens coatings, coatings for surgical implants and medical devices, as gels, and as ingredients in pharmaceutical solutions including delayed-release pharmaceutical formulations and targeted pharmaceutical formulations.

### BRIEF DESCRIPTION OF THE DRAWINGS

FIG. 1 shows a schematic representation of a reaction of the invention.

FIG. 2 shows a schematic representation of a reaction of the invention.

FIG. 3 shows a schematic representation of a reaction of the invention.

FIG. 4 shows the chemical structure of ibuprofen, with a diene attached thereto.

FIG. 5 shows the chemical structure of naproxen, with a diene attached thereto.

FIG. 6 shows the chemical structure of 2-(undec-10-enyl)tridec-12-enoic acid.

FIG. 7 shows the chemical structure of 2-(undec-10-enyl)tridec-12-en-1-ol.

FIG. 8 shows the chemical structure of (S)-2-(undec-10-enyl)tridec-12-enyl 2-(4-isobutylphenyl)propanoate.

FIG. 9 shows the chemical structure of (S)-2-(undec-10-enyl)tridec-12-enyl 2-(6-methoxynapthalen-2-yl)propanoate.

FIG. 10 shows the chemical structure of (S)-2-(2-(2-(2-hydroxyethoxy)ethoxy)ethoxy)ethyl 2-(6-methoxynaphthalen-2-yl)propanoate.

FIG. 11 shows the chemical structure of (S)-10-hydroxydecyl 2-(4-isobutylphenyl)propanoate.

FIG. 12 shows the chemical structure of (S)-2-(2-(2-(2-hydroxyethoxy)ethoxy)ethoxy)ethyl 2-(6-methoxynaphthalen-2-yl)propanoate.

FIG. 13 shows the chemical structure of (S)-10-hydroxydecyl 2-(6-methoxynaphthalen-2-yl)propanoate.

FIG. 14 shows the chemical structure of (S)-14-(4-isobutylphenyl)-13-oxo-3,6,9,12-tetroxapentadecyl 2-(undec-10-enyl)tridec-12-enoate.

FIG. 15 shows the chemical structure of (S)-10-(2-(4-isobutylphenyl)propanoyloxy)decyl 2-(undec-10-enyl)tridec-12-enoate.

FIG. 16 shows the chemical structure of (S)-14-(6-methoxynaphthalen-2-yl)-13-oxo-3,6,9,12-tetroxapentadecyl 2-(undec-10-enyl)tridec-12-enoate.

FIG. 17 shows the chemical structure of (S)-10-(2-(6-methoxynaphthalen-2-yl)propanoyloxy)decyl 2-(undec-10-enyl)tri dec-12-enoate.

FIG. 18 shows diverging schemes, illustrating distinctions between a prior approach (top scheme) and embodiments of methods of the invention (lower scheme).

FIG. 19 shows the chemical structure of a polymer of the invention, wherein the biologically active molecule is an antibiotic.

FIG. 20 shows the chemical structure of a polymer of the invention, wherein the biologically active molecule is an analgesic.

FIG. 21 shows the chemical structure of a polymer of the invention, wherein the biologically active molecule is an antibacterial compound.

### DETAILED DESCRIPTION OF THE INVENTION

The present invention concerns an acyclic diene metathesis (ADMET) chemistry-based method of making polymers incorporating biologically active (bioactive) molecules, and the polymers formed thereby. The invention provides a step-growth polymerization technique that allows both the precise predetermination of polymer architecture and control of drug-loading. The polydispersity of a step-growth polymerization was predicted to be 2, and the inventors are able to confirm this experimentally.

The polymers of the invention comprise a covalently attached biologically active molecule (e.g., a drug) covalently attached (in a variable amount) through monomer design. This family of polymer materials has a very well known primary polymer structure. In the methods of the invention, a diene molecule functionalized with a bioactive molecule, or chain thereof, is used as a monomer that is polymerized by ADMET.

Advantageously, the polymer of the invention can be produced in a manner in which the following is controlled (predetermined): (a) the amount of the bioactive agent; (b) the architecture of the resulting polymer (i.e., the specific location of the bioactive molecules on the polymer); and (c) how fast the bioactive agent is released or exposed. Furthermore, depending on the amount of residual catalyst remaining in the polymer after synthesis, these materials can be nearly non-toxic.

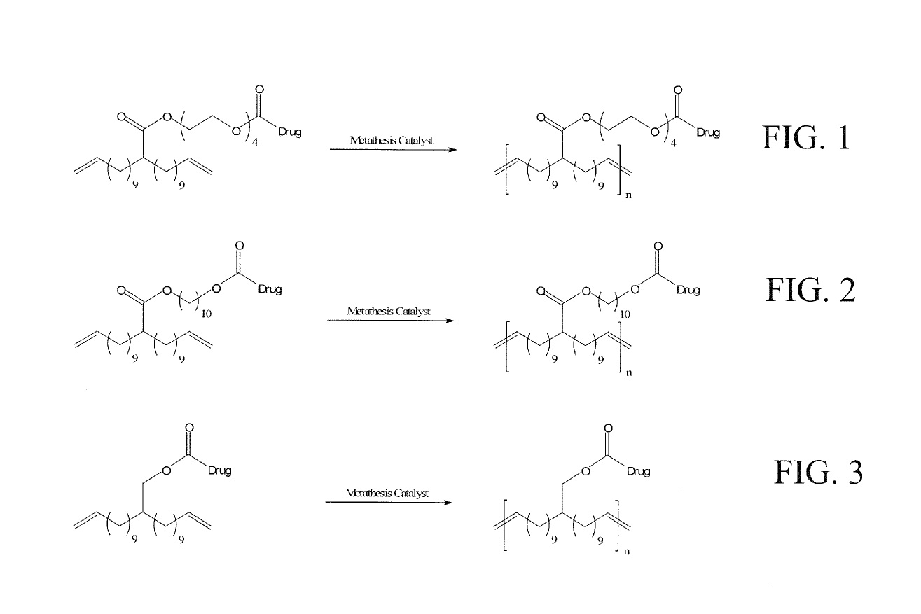

The polymers of the invention can be prepared by ADMET chemistry utilizing a suitable catalyst as illustrated in FIGS. 1-3. In general, the reactions include two steps. The method comprises producing or providing biologically active molecule (drug)-branched diene monomers, and using ADMET to polymerize the monomers into a polymer product. Using the method, polyolefin polymers having bioactive molecules positioned at precise locations pendant to the backbone are produced. The bioactive molecules can be incorporated into the monomers and polymers of the invention by various linkages (e.g., decanediol ester drugs, tetraethylene glycol ester drugs, etc.). Thus, in addition to other subject matter, the invention provides biologically active molecule-functionalized polymers; (2) methods of making such polymers; and (3) products incorporating such polymers. Examples of products incorporating one or more polymers of the invention as components include biomaterials designed and constructed to be placed in or onto the body, or to contact fluid or tissue of the body. Products incorporating one or more polymers of the invention can be medical devices that have one or more surfaces that contact blood or other bodily tissues in the course of their operation, such as vascular grafts, stents, heart valves, orthopedic devices, catheters, shunts, and the like.

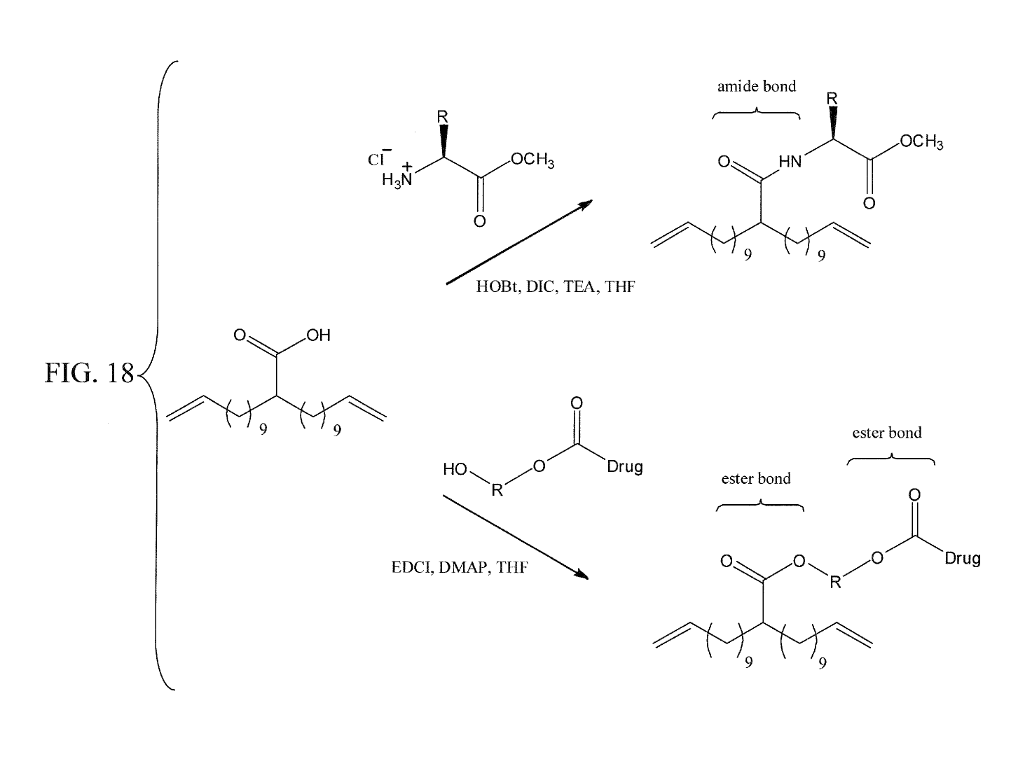

FIG. 18 shows two diverging schemes, illustrating distinctions between a prior method (upper scheme) and certain embodiments of methods of the invention (lower scheme). Both schemes of FIG. 18 are initiated with a diene acid. The 9,9 acid is shown in FIG. 18; however, one skilled in the art would appreciate that other starting materials such as the 3,3 acid, 6,6 acid, or 18,18 acid may be used, for example. The two approaches diverge at the point where the carboxylic acid functionality of the diene is covalently linked. At the top right of FIG. 18, an amino acid (or a polypeptide) is added through a stable amide bond (as labeled). The amide/peptide bond is very stable to chemical hydrolysis and is readily cleaved by amidase enzymes, which are generally not present in the blood or extracellular fluid of the body; thus these bio-olefin materials in the top scheme of FIG. 18 are generally stable in the body (not readily degrading).

The product of the lower scheme (lower right of FIG. 18) is a polymer prodrug. In generally, a prodrug is unreactive and is metabolized to the biologically active form (or more biologically active form), i.e., to the active pharmaceutical species, in the body. In some embodiments of the invention (including that shown in FIG. 18, lower scheme), the materials are designed to have two ester linkages available for hydrolysis (cleavage). It is advantageous to obtain a polymer prodrug that is stable enough to assemble and reach the intended anatomical site (target), yet reactive enough to be readily cleaved off when it is at the target site. The “R” group in the lower scheme in FIG. 18, between the two oxygen atoms, is the spacer and can be varied to be long or short, and can be hydrophobic or hydrophilic, for example, depending upon the desired properties. Advantageously, the polymer materials of the invention can be designed to degrade in the body at a controlled rate through cleavable linkages.

In one embodiment, the polymer of the invention is a prodrug, wherein the bioactive molecule is therapeutic. In this and other embodiments, the polymer may be formed as a coating, solution, gel, nanoparticle (e.g., nanosphcre), microparticle (e.g., microsphere), or other formulation appropriate for the intended application.

The embodiments described herein illustrate adaptations of the methods and compositions of the invention. Nonetheless, from the description of these embodiments, other aspects of the invention can also be made and/or practiced.

### General Methods

The method of the invention can utilize general techniques known in the field of polymer chemistry. General polymer chemistry concepts and methods that may be utilized are described in the Polymer Handbook (4^{th} Edition), eds., Brandup et al., New York, John Wiley and Sons, 1999; and Polymer Synthesis and Characterization: A Laboratory Manual, eds. Sandler et al., Academic Press, 1998. Concepts and methods relating more specifically to metathesis chemistry are described in Alkene Metathesis in Organic Synthesis. Springer-Verlag: Berlin, 1998 and Olefin Metathesis and Metathesis Polymerization, 2nd ed.; Academic: San Diego, 1997. ADMET is described with particularity in Lindmark-Hamburg, M. and Wagener, K. B. Macromolecules 1987, 20:2949; Wagener et al., Macromolecules 1990, 23:5155; Smith et al., Macromolecules 2000, 33:3781-3794; Watson, M. D. and Wagener, K. B., Macromolecules, 2000, 33:3196-3201; Watson M. D. and Wagener K. B., Macromolecules, 2000, 33:8963-8970; and Watson M. D. and Wagener K. B. Macromolecules, 2000, 33:5411-5417.

### Monomers

In methods of the invention, a diene molecule functionalized with a bioactive molecule, or chain thereof, is used as a monomer that is polymerized by ADMET. Any type of diene molecule functionalized with a bioactive molecule (or chain thereof) that is capable of being polymerized by the metathesis method taught herein may be used as the monomer. Two or more (e.g., 3, 4, 5, 6, 7, 8 or more) different monomers of this type may also be used to produce co-polymers.

By using a diene, such as 3,3 acid, 6,6 acid, 9,9 acid, or 18,18 acid monomers, the amount of biologically active molecule (drug) on the polymer backbone can be varied at exact intervals and thereby controlled. For example, the 3,3 acid will result in a drug molecule on every ninth carbon for the resultant polymer, the 6,6 acid would yield a drug molecule every fifteenth carbon, the 9,9 acid would yield a drug molecule every twenty-first carbon, and the 18,18 acid would provide a drug molecule every thirty-ninth carbon on the backbone. The formula for this design is 2n+2, with n being the spacer. Having the capability to vary the drug-loading while still simultaneously knowing the exact placement of the drug molecules is a huge benefit for a drug delivery material.

In the examples described below, dienes functionalized with a bioactive molecule (ibuprofen, naproxen) pendant to the backbone of the diene are used. For a description of alkyne metathesis chemistry see, e.g., Zhang et al., Youji Huaxue, 2001, 21:541-548; Winfried et al., Eur. J. Chem., 2001, 7:117-126; and Brizius et al., J. Am. Chem. Soc., 2000, 122:12435-12440. Preferred conditions for condensing such other molecules can be identified by performing the reactions described below under various reaction conditions to identify those under which a particular reaction proceeds efficiently. The conditions described herein can be used as a general guide in setting the ranges of the reaction conditions to be tested. In the experiments described below, standard ACS reagent grade chemicals were used as substrates and are commercially available.

### Catalysts

The ADMET-mediated condensation of a diene according to the invention is facilitated using a metathesis catalyst. Any methathesis catalyst compatible with the methods of the invention may be used. Preferably, metathesis catalysts tolerant to the wide variety of functional groups found in drug molecules and the linkers that connect them to the polymer backbone are utilized. In one embodiment, the catalyst is a ruthenium-based catalyst, such as those found in Grubbs “First Generation” or “Second Generation” catalysts. Hoyveda's catalysts or modifications to these ruthenium (Ru)-based materials may improve the activity and tolerance of these catalysts.

Numerous ADMET catalysts are known. Many of these, however, are not suitable for use with functionalized monomers as the functional groups can interfere with the active site of the catalyst molecule. For this reason, ADMET catalysts known to be tolerant of functional groups may be preferred. A tungsten halide in combination with an aluminum alkyl (e.g., tungsten hexachloride and ethyl aluminum dichloride) may be effective. However, the molybdenum- and tungsten-based metathesis catalysts are less preferred due to their extreme reactivity to functional groups.

Because of their well-known tolerance of functional groups and efficiency of catalysis, Ru-based catalysts are useful in the reactions of the invention. For example, 1,3-dimesityl-4,5-dihydroimidazol-2-ylidene)benzylidene ruthenium dichloride is useful because of its ability to efficiently catalyze the exemplary reactions described below. Scholl et al., Org. Lett., 1999, 1:953). However, the ADMET catalyst can be any of a variety of catalysts capable of effecting metathesis polymerization. Examples include Schrock's molybdenum alkylidene catalyst, Grubbs' ruthenium benzylidene catalyst, and Grubbs' imidazolium catalyst (“Super-Grubbs”). A number of other catalysts may be employed in the reaction was well.

### Linkers and Spacers

Regardless of the particular bioactive active molecule(s) intended to be used, the reactive functional groups should be protected for the polymerization process. Whatever protection group is used on the bioactive molecule for polymerization, unless it can be cleaved naturally by the body, it should be removed via deprotection chemistry in order to have its therapeutic effect in the body. Ester bonds and the tetraethylene and decanediol spacers have been used in some of the examples due to their availability; however, many more spacers and linkages can be used.

A large variety of linkers may be utilized in the polymers and methods of the invention. In a preferred embodiment, ester linkages are utilized. Depending upon the rate at which these linkers are cleaved in the body, they can be modified to cleave either faster or more slowly. For example, if relatively slower cleavage is desirable, a carbamate, carbonate, or even an amide linker can be utilized. More rapid cleavage could also be achieved by retaining the ester but adding strong electron-withdrawing groups alpha to the ester. The increased pull of electrons from the ester carbon will increase the rate at which it hydrolyzes.

The spacer(s) can be selected to make cleavage of the bioactive molecule in the body more rapid or less rapid. Spacers may be more hydrophobic or more hydrophilic, for example, depending upon the desired properties. Generally, a hydrophobic spacer (e.g., glycols) would be expected to increase enzymatic hydrolysis while slowing chemical hydrolysis. Conversely, a hydrophilic spacer would generally be expected to decrease enzymatic hydrolysis but increase chemical hydrolysis. Lack of a spacer would likely minimize enzymatic hydrolysis but still allow some chemical hydrolysis.

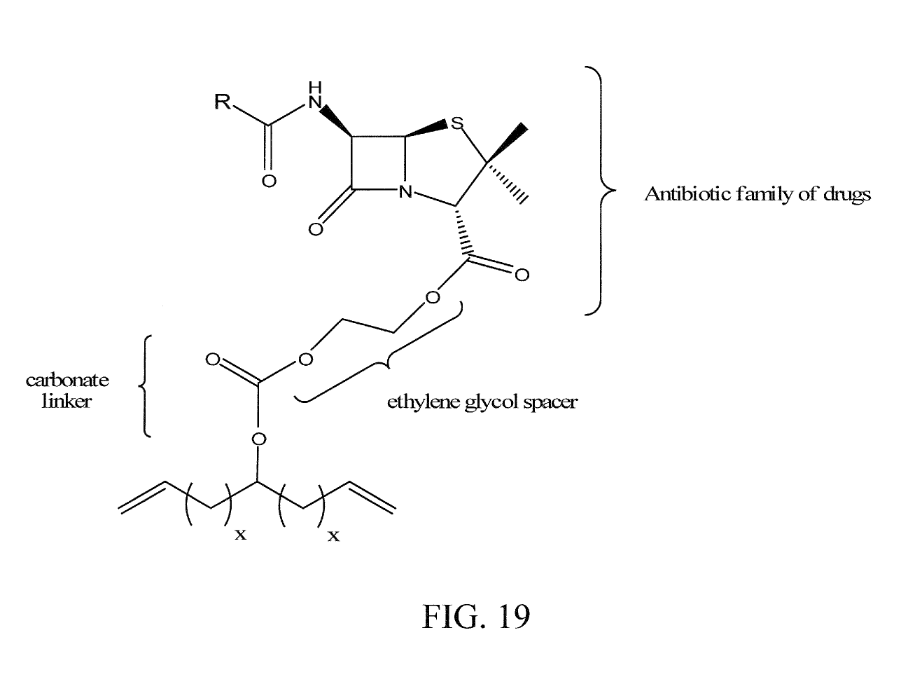

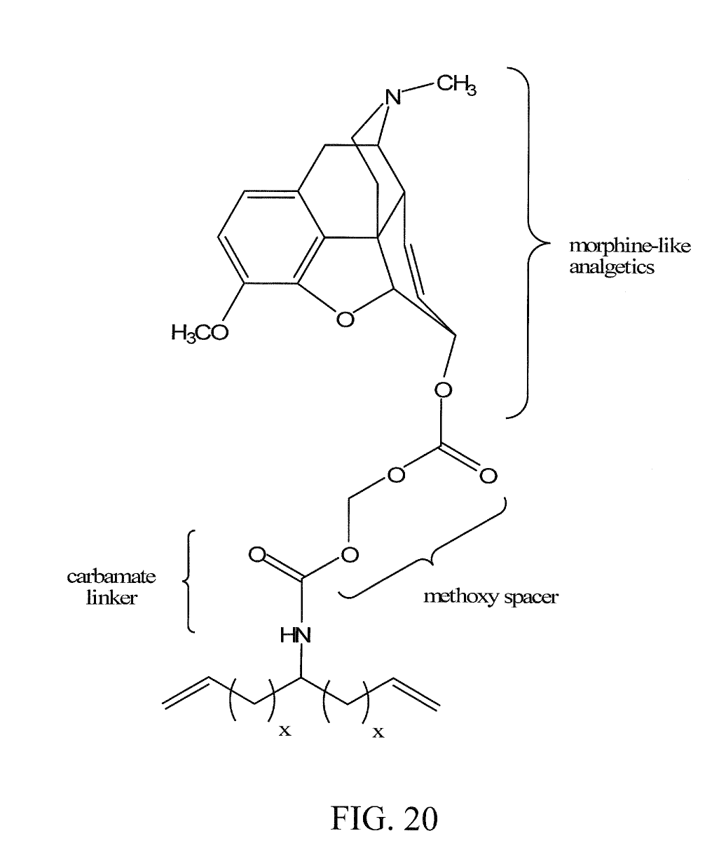

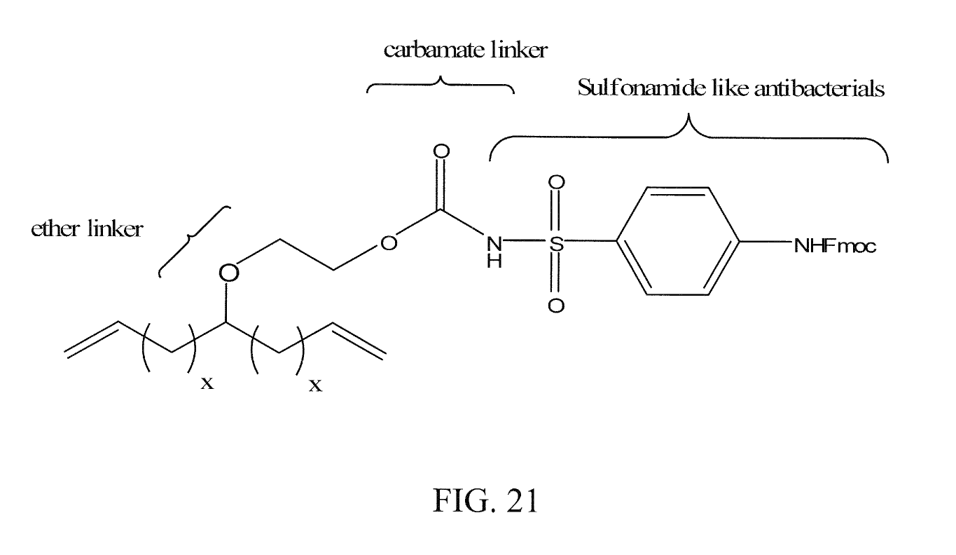

As described herein, any biologically active molecule can be used in the polymers and methods of the invention. The polymers and methods of the invention are particularly advantageous for delivery of potent drugs that are quickly metabolized by the body. For example, analgetics (such as morphine) and antibacterials and antibiotics (such as tetracyclines) can be used. FIGS. 19-21 show the chemical structures of polymers of the invention, bearing antibiotic, analgesic, and antibacterial compounds, respectively. The various linkers described herein are compatible with these examples of drugs. Carbonate, carbamate, ether, and ester linkages each degrade at different rates in the body. Although various spacers can be selected in designing various embodiments of polymers of the invention, preferred spacers include methoxy spacers and glycol spacers (e.g., ethylene glycol spacer). Various combinations of linkers and spacers may be used (e.g., carbonate linker and ethylene glycol spacer; carbamate linker and methoxy spacer; ether linker and carbamate linker, etc.).

In one embodiment, the linker is not an amide (a non-amide moiety). In another embodiment, the bioactive molecule is not connected to the spacer or linker through an amino acid or peptide. In another embodiment, the linker is not an amide and the bioactive molecule is not connected to the space or linker through an amino acid or peptide.

### Pharmaceutical Compositions

The subject invention includes pharmaceutical compositions comprising polymers of the invention that contain bioactive molecules, in association with a pharmaceutically acceptable carrier. The pharmaceutical compositions of the subject invention can be formulated according to known methods for preparing pharmaceutically useful compositions. The carrier may be liquid, solid, or semi-solid, for example. Formulations are described in a number of sources which are well known and readily available to those skilled in the art. For example, Remington's Pharmaceutical Sciences (Martin E W, 1995, Easton Pa., Mack Publishing Company, 19^{th} ed.) describes formulations which can be used in connection with the subject invention. Formulations suitable for parenteral administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents.

The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc. It should be understood that in addition to the ingredients particularly mentioned above, the formulations of the subject invention can include other agents conventional in the art having regard to the type of formulation in question.

Administration of the polymers of the subject invention to cells in vitro or to a human or non-human animal subject can be achieved by conventional procedures known by those of ordinary skill in the art and disclosed in the literature. Aqueous solutions of polymers are most conveniently used. Administration may be achieved by any route or method. For example, the polymers (and compositions comprising the polymers) can be administered parentally, such as by intravenous administration. One of skill in the art can readily determine appropriate dosages, concentrations, and rates and duration of administration, based on the size of the subject and the route of administration.

In yet another aspect, the subject invention is directed to methods for the administration of polymers of the invention, which are prepared in accordance with the subject invention, to a human or non-human animal cell in vitro or in vivo in a pharmaceutically effective amount. The methods of administration further comprise providing a polymer prepared in accordance with the subject invention and contacting a target cell with an effective amount of the polymer. In one specific embodiment, the polymer is administered within a pharmaceutically acceptable carrier.

Methods of administration include, but are not limited to, intra-arterial, intramuscular, intravenous, intranasal, topical, and oral routes. In a specific embodiment, the pharmaceutical compositions of the invention can be administered locally to the area in need of treatment; such local administration can be achieved, for example, by local infusion during surgery, by injection, by topical application, or by means of a catheter.

Therapeutic amounts of polymers (or bioactive molecules contained therein) can be empirically determined and will vary with the pathology being treated, the subject being treated, and the efficacy and toxicity of the bioactive agent contained within the polymer. Similarly, suitable dosage formulations and methods of administering the bioactive molecules can be readily determined by those of skill in the art.

The polymers of the invention can be administered by any of a variety of routes, such as orally, intranasally, parenterally or by inhalation therapy, and can take the form of tablets, lozenges, granules, capsules, pills, ampoule, suppositories or aerosol form. They can also take the form of suspensions, solutions, and emulsions of the active ingredient in aqueous or nonaqueous diluents, syrups, granulates or powders. In addition to the bioactive molecules specifically identified herein, the polymers of the invention can also contain other pharmaceutically active compounds or a plurality of compounds.

The invention encompasses co-administration steps, with co-administration amounts, or with both the steps and the amounts together, which provide the desired pharmaceutical effect. Advantages of such co-administration can include improvement in the side-effect profiles of one or more of the co-administered agents. There are significant amounts of esterases in blood and tissues. However, in some embodiments in which it is not desired to rely on the body's endogenous enzymes or chemical hydrolysis mechanisms to cleave off or expose the bioactive molecule at the site of the spacer and/linker of the polymer, one or more enzymes effective at cleaving off or exposing the bioactive molecule of the polymer may be administered to the subject before, during, or after administration of the polymer of the invention. The administered enzyme(s) may be a type naturally produced in the subject (endogenous in the patient) or a type that is not naturally produced in the subject (heterologous).

Advantageously, the polymers of the invention can be administered simultaneously or sequentially with other polymers, drugs, or other biologically active agents. Examples include, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids.

The polymers of the invention are useful for making articles of manufacture including devices (e.g., implantable or deployable medical devices), and coatings for releasing biologically active molecules (optionally, the molecules may be beneficial, e.g., therapeutic). The polymers of the invention can be processed into articles, including delivery devices, and/or coated onto a substrate by standard manufacturing techniques. For example, the polymers of the invention can be extruded into filaments, pressed into shaped articles, solvent film cast, doctor-bladed into thin films, coated onto a substrate by solvent evaporation, compression and transfer molded, and processed by like standard methods of manufacture. Other devices provided by the invention include a device for the controlled release of a biologically active molecule (biologically active agent) wherein the device is a matrix of the polymer having a biologically active agent present in the matrix with the device eroding and releasing the agent over time.

The polymers of the invention can be used as a single film, or in a number of layers made of different polymers of this invention, and they can be made into devices of various geometric shapes, for example, flat, square, round, tubular, disc, ring, and the like. Furthermore, the devices of the invention can be sized, shaped, and adapted for implantation, insertion or placement on the body, in the body, its cavities and passageways, or for positioning in other environments for example, fields or reservoirs. The polymers are useful for making devices for dispensing a biologically active molecule (biologically active agent) and for use as coatings as they erode with an accompanying dispensing of the agent.

The polymers are useful in embodiments for manufacturing polymeric delivery compositions containing a biologically active molecule (drug) which composition erodes in vivo with an accompanying release of the drug. Many variations of compositions and delivery devices will be apparent to those skilled in the art of dispensing agents in the light of this invention. For example, a number of layers can be used, a variety of agents, including drugs, can be used in several layers, and polymers having different erosion rates can be used for obtaining different delivery patterns.

In another aspect, the polymers of the invention are useful for coating agents that lend themselves to use as slow release fertilizers. The fertilizers can be coated in their conventional forms such as granules, powder, beads, particles, and the like. Fertilizers that can be coated include urea, fertilizers with slow ammonia release, fertilizers in the form of water soluble salts, which salts contain nitrogen, phosphorous, and sulfur, potassium, calcium, magnesium, manganese, zinc, copper, boron, and the like. Also, fertilizers such as the common fertilizers designated by 8-24-12, 8-8-6, 5-20-20, 12-12-12, 14-16-0, 8-4-6, 3-9-6, and the like can be coated. Additionally, the fertilizer or plant nutrient can be impregnated into, or suitably admixed with inert materials, such as silica. The coating compositions can additionally contain pigments, dyes, driers, stabilizers and the like.

The practice of the subject invention can employ, unless otherwise indicated, conventional techniques of molecular biology, microbiology, recombinant DNA technology, electrophysiology, and pharmacology, which are within the ordinary skill in the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover ed. 1985); Perbal, B., A Practical Guide to Molecular Cloning (1984); the series, Methods In Enzymology (S. Colowick and N. Kaplan eds., Academic Press, Inc.); Transcription and Translation (Hames et al. eds. 1984); Gene Transfer Vectors For Mammalian Cells (J. H. Miller et al. eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. eds. (1991) IRL Press)).

### EXEMPLIFIED EMBODIMENTS

### Embodiment 1

A polymer comprising a plurality of repeating diene monomers having coupled thereto at least one biologically active molecule through at least one non-amide linking moiety (linker).

### Embodiment 2

The polymer of embodiment 1, wherein the linker is least one moiety selected from the group consisting of an ester, carbonate, carbomate, and ether.

### Embodiment 3

The polymer of embodiment 1 or 2, wherein linker comprises an ether moiety and carbamate moiety.

### Embodiment 4

The polymer of embodiment 1-3, wherein the diene monomer is selected from the group consisting of 3,3 acid, 6,6 acid, 9,9 acid, and 18,18 acid.

### Embodiment 5

The polymer of any of embodiments 1-4, wherein the monomer further comprises at least one spacer interposed between the biologically active molecule and the linker.

### Embodiment 6

The polymer of embodiment 5, wherein the spacer is at least one moiety selected from the group consisting of methoxy and glycol.

### Embodiment 7

The polymer of embodiment 6, wherein the spacer is at least one ethylene glycol moiety.

### Embodiment 8

The polymer of embodiment 6, wherein the spacer is diethylene glycol or triethylene glycol.

### Embodiment 9

The polymer of any of embodiments 1-5, wherein the spacer is at least one alkyl or diol moiety.

### Embodiment 10

The polymer of any of embodiments 1-9, wherein the at least one linker, the at least one spacer, or both are degradable by enzymatic cleavage, chemical hydrolysis, or both.

### Embodiment 11

The polymer of any of embodiments 1-10, wherein the biologically active molecule is selected from the group consisting of analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, pesticides, prostaglandins, radio-pharmaceuticals, hormones, sex hormones (including steroids), time release hinders, anti-allergic agents, stimulants and anoretics, steroids, sympathomimetics, thyroid agents, vaccines, vasodilators, and xanthines.

### Embodiment 12

A pharmaceutical composition comprising a polymer of any of embodiments 1-11; and a pharmaceutically acceptable carrier.

### Embodiment 13

A method for making a polymer of any of embodiments 1-11, comprising providing diene monomers coupled to at least one biologically active molecule through at least one non-amide linking moiety (linker); and polymerizing the monomers to produce the polymer.

### Embodiment 14

The method of embodiment 13, wherein said providing comprises coupling the at least one biologically active molecule to the diene through at least one linker.

### Embodiment 15

A method for delivering a biologically active molecule to a subject, comprising administering a polymer of any of embodiments 1-11 to the subject.

### Embodiment 16

A device comprising a polymer of any of embodiments 1-11.

### Embodiment 17

The device of embodiment 16, wherein the device comprises one or more substrates (surfaces) coated with the polymer or coated with a composition comprising the polymer.

### Embodiment 18

The device of embodiment 16, wherein the device is an implantable or deployable medical device.

The following definitions are used, unless otherwise described.

As used herein, the term “drug” is interchangeable with the term “biologically active molecule” or “bioactive molecule” and refers to any agent capable of having a physiologic effect (e.g., a therapeutic or prophylactic effect, toxic effect, etc.) on a biosystem such as prokaryotic or eukaryotic cells, in vivo or in vitro, including, but without limitation, chemotherapeutics, toxins, radiotherapeutics, radiosensitizing agents, gene therapy vectors, antisense nucleic acid constructs, transcription factor decoys, imaging agents, diagnostic agents, agents known to interact with an intracellular protein, polypeptides, and polynucleotides. Drugs that may be utilized in the polymers of the invention include any type of compound, such as antibacterial, antiviral, antifungal, or anti-cancer agents, that can be coupled to a polymerizable monomer moiety (producing a polymer-drug conjugate).

Although the experiments in the Examples section were carried out with non-steroidal anti-inflammatory drugs (i.e., naproxen and ibuprofen), the methods of the invention can be used to produce polymers bearing other drugs with controlled functionality. The drug can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The bioactive molecule need not be a therapeutic agent. For example, the bioactive molecule may be cytotoxic to the local cells to which it is delivered but have an overall beneficial effect on the subject. Further, the bioactive molecule may be a diagnostic agent with no direct therapeutic activity per se, such as a contrast agent for bioimaging.

A description of these classes of drugs and diagnostic agents and a listing of species within each class can be found, for instance, in Martindale, The Extra Pharmacopoeia, Twenty-ninth Edition (The Pharmaceutical Press, London, 1989), which is incorporated herein by reference in its entirety. Drugs or diagnostic agents are commercially available and/or can be prepared by techniques known in the art.

Poorly water soluble drugs which may be used in the practice of the subject invention include but are not limited to alprazolam, amiodarone, amlodipine, astemizole, atenolol, azathioprine, azelatine, beclomethasone, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyridamole, divalproex, dobutamine, doxazosin, enalapril, estradiol, etodolac, etoposide, famotidine, felodipine, fentanyl citrate, fexofenadine, finasteride, fluconazole, flunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, phenyloin, piroxicam, quinapril, ramipril, risperidone, sertraline, simvastatin, sulindac, terbinafine, terfenadine, triamcinolone, valproic acid, zolpidem, or pharmaceutically acceptable salts of any of the above-mentioned drugs.

The bioactive molecule of the polymers of the invention can be an anti-cancer agent. As used herein, the term “anti-cancer agent” refers to a substance or treatment that inhibits the function of cancer cells, inhibits their formation, and/or causes their destruction in vitro or in vivo. Examples include, but are not limited to, cytotoxic agents (e.g., 5-fluorouracil, TAXOL), chemotherapeutic agents, and anti-signaling agents (e.g., the PI3K inhibitor LY). In some embodiments, the anti-cancer agent is a ras antagonist.

The bioactive molecule of the polymers of the invention can be a cytotoxic agent. As used herein, the term “cytotoxic agent” refers to a substance that inhibits or prevents the function of cells and/or causes destruction of cells in vitro and/or in vivo. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³², and radioactive isotopes of Lu), chemotherapeutic agents, toxins such as small molecule toxins or enzymatically active toxins of bacterial, fungal, plant or animal origin, and antibodies, including fragments and/or variants thereof.

The bioactive molecule of the polymers of the invention can be a chemotherapeutic agent. As used herein, the term “chemotherapeutic agent” is a chemical compound useful in the treatment of cancer, such as, for example, taxanes, e.g., paclitaxel (TAXOL, BRISTOL-MYERS SQUIBB Oncology, Princeton, N.J.) and doxetaxel (TAXOTERE, Rhone-Poulenc Rorer, Antony, France), chlorambucil, vincristine, vinblastine, anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON, GTx, Memphis, Tenn.), and anti-androgens such as flutamide, nilutamide, bicalutamide, leuprolide, and goserelin, etc. Examples of chemotherapeutic agents that may be used as the bioactive component in the polymeric materials of the invention are listed in Table 1. For example, the chemotherapeutic agent may be one or more anthracyclines. Anthracyclines are a family of chemotherapy drugs that are also antibiotics. The anthracyclines act to prevent cell division by disrupting the structure of the DNA and terminate its function by: (1) intercalating into the base pairs in the DNA minor grooves; and (2) causing free radical damage of the ribose in the DNA. The anthracyclines are frequently used in leukemia therapy. Examples of anthracyclines include daunorubicin (CERUBIDINE), doxorubicin (ADRIAMYCIN, RUBEX), epirubicin (ELLENCE, PHARMORUBICIN), and idarubicin (IDAMYCIN).

TABLE 1
Examples of Chemotherapeutic Agents

| | |
|---|---|
| 13-cis-Retinoic Acid | Mylocel |
| 2-Amino-6- | Letrozole |
| Mercaptopurine | Neosar |
| 2-CdA | Neulasta |
| 2-Chlorodeoxyadenosine | Neumega |
| 5-fluorouracil | Neupogen |
| 5-FU | Nilandron |
| 6-TG | Nilutamide |
| 6-Thioguanine | Nitrogen Mustard |
| 6-Mercaptopurine | Novaldex |
| 6-MP | Novantrone |
| Accutane | Octreotide |
| Actinomycin-D | Octreotide acetate |
| Adriamycin | Oncospar |
| Adrucil | Oncovin |
| Agrylin | Ontak |
| Ala-Cort | Onxal |
| Aldesleukin | Oprevelkin |
| Alemtuzumab | Orapred |
| Alitretinoin | Orasone |
| Alkaban-AQ | Oxaliplatin |
| Alkeran | Paclitaxel |
| All-transretinoic acid | Pamidronate |
| Alpha interferon | Panretin |
| Altretamine | Paraplatin |
| Amethopterin | Pediapred |
| Amifostine | PEG Interferon |
| Aminoglutethimide | Pegaspargase |
| Anagrelide | Pegfilgrastim |
| Anandron | PEG-INTRON |
| Anastrozole | PEG-L-asparaginase |
| Arabinosylcytosine | Phenylalanine Mustard |
| Ara-C | Platinol |
| Aranesp | Platinol-AQ |
| Aredia | Prednisolone |
| Arimidex | Prednisone |
| Aromasin | Prelone |
| Arsenic trioxide | Procarbazine |
| Asparaginase | PROCRIT |
| ATRA | Proleukin |
| Avastin | Prolifeprospan 20 with Carmustine implant |
| BCG | Purinethol |
| BCNU | Raloxifene |
| Bevacizumab | Rheumatrex |
| Bexarotene | Rituxan |
| Bicalutamide | Rituximab |
| BiCNU | Roveron-A (interferon alfa-2a) |
| Blenoxane | Rubex |
| Bleomycin | Rubidomycin hydrochloride |
| Bortezomib | Sandostatin |
| Busulfan | Sandostatin LAR |
| Busulfex | Sargramostim |
| C225 | Solu-Cortef |
| Calcium Leucovorin | Solu-Medrol |
| Campath | STI-571 |
| Camptosar | Streptozocin |
| Camptothecin-11 | Tamoxifen |
| Capecitabine | Targretin |
| Carac | Taxol |
| Carboplatin | Taxotere |
| Carmustine | Temodar |
| Carmustine wafer | Temozolomide |
| Casodex | Teniposide |
| CCNU | TESPA |
| CDDP | Thalidomide |
| CeeNU | Thalomid |
| Cerubidine | TheraCys |
| cetuximab | Thioguanine |
| Chlorambucil | Thioguanine Tabloid |
| Cisplatin | Thiophosphoamide |
| Citrovorum Factor | Thioplex |
| Cladribine | Thiotepa |
| Cortisone | TICE |
| Cosmegen | Toposar |
| CPT-11 | Topotecan |
| Cyclophosphamide | Toremifene |
| Cytadren | Trastuzumab |
| Cytarabine | Tretinoin |
| Cytarabine liposomal | Trexall |
| Cytosar-U | Trisenox |
| Cytoxan | TSPA |
| Dacarbazine | VCR |
| Dactinomycin | Velban |
| Darbepoetin alfa | Velcade |
| Daunomycin | VePesid |
| Daunorubicin | Vesanoid |
| Daunorubicin | Viadur |
| hydrochloride | Vinblastine |
| Daunorubicin liposomal | Vinblastine Sulfate |
| DaunoXome | Vincasar Pfs |
| Decadron | Vincristine |
| Delta-Cortef | Vinorelbine |
| Deltasone | Vinorelbine tartrate |
| Denileukin diftitox | VLB |
| DepoCyt | VP-16 |
| Dexamethasone | Vumon |
| Dexamethasone acetate | Xeloda |
| dexamethasone sodium | Zanosar |
| phosphate | Zevalin |
| Dexasone | Zinecard |
| Dexrazoxane | Zoladex |
| DHAD | Zoledronic acid |
| DIC | Zometa |
| Diodex | Gliadel wafer |
| Docetaxel | Glivec |
| Doxil | GM-CSF |
| Doxorubicin | Goserelin |
| Doxorubicin liposomal | granulocyte-colony stimulating factor |
| Droxia | Granulocyte macrophage colony stimulating |
| DTIC | factor |
| DTIC-Dome | Halotestin |
| Duralone | Herceptin |
| Efudex | Hexadrol |
| Eligard | Hexalen |
| Ellence | Hexamethylmelamine |
| Eloxatin | HMM |
| Elspar | Hycamtin |
| Emcyt | Hydrea |
| Epirubicin | Hydrocort Acetate |
| Epoetin alfa | Hydrocortisone |
| Erbitux | Hydrocortisone sodium phosphate |
| Erwinia L-asparaginase | Hydrocortisone sodium succinate |
| Estramustine | Hydrocortone phosphate |
| Ethyol | Hydroxyurea |
| Etopophos | Ibritumomab |
| Etoposide | Ibritumomab Tiuxetan |
| Etoposide phosphate | Idamycin |
| Eulexin | Idarubicin |
| Evista | Ifex |
| Exemestane | IFN-alpha |
| Fareston | Ifosfamide |
| Faslodex | IL-2 |
| Femara | IL-11 |
| Filgrastim | Imatinib mesylate |
| Floxuridine | Imidazole Carboxamide |
| Fludara | Interferon alfa |
| Fludarabine | Interferon Alfa-2b (PEG conjugate) |
| Fluoroplex | Interleukin-2 |
| Fluorouracil | Interleukin-11 |
| Fluorouracil (cream) | Intron A (interferon alfa-2b) |
| Fluoxymesterone | Leucovorin |
| Flutamide | Leukeran |
| Folinic Acid | Leukine |
| FUDR | Leuprolide |
| Fulvestrant | Leurocristine |
| G-CSF | Leustatin |
| Gefitinib | Liposomal Ara-C |
| Gemcitabine | Liquid Pred |
| Gemtuzumab ozogamicin | Lomustine |
| Gemzar | L-PAM |
| Gleevec | L-Sarcolysin |
| Lupron | Meticorten |
| Lupron Depot | Mitomycin |
| Matulane | Mitomycin-C |
| Maxidex | Mitoxantrone |
| Mechlorethamine | M-Prednisol |
| Mechlorethamine | MTC |
| Hydrochlorine | MTX |
| Medralone | Mustargen |
| Medrol | Mustine |
| Megace | Mutamycin |
| Megestrol | Myleran |
| Megestrol Acetate | Iressa |
| Melphalan | Irinotecan |
| Mercaptopurine | Isotretinoin |
| Mesna | Kidrolase |
| Mesnex | Lanacort |
| Methotrexate | L-asparaginase |
| Methotrexate Sodium | LCR |
| Methylprednisolone | |

One skilled in the art will appreciate that the terms “anti-cancer agent,” “cytotoxic agent,” and “chemotherapeutic agent” are not mutually exclusive (i.e., overlap exists).

The biologically active molecules in the polymers of the invention may also be pesticides, herbicides, germicides, biocides, algicides, rodenticides, fungicides, insecticides, plant growth promoters, plant growth inhibitors, preservatives, disinfectants, sterilization agents, cosmetics, plant foods, fertilizers, vitamins, sex sterilants, plant hormones, fertility inhibitors, fertility promoters, air-purifiers, micro-organism attenuators, nutrients and the like.

The biologically active molecules in the polymers of the invention can be local and systemic drugs that produce a physiologic and pharmacologic beneficial result in animals, avians, reptiles and fish. The term “animals” is inclusive of mammals, which includes human and non-human mammals. The drugs may be inorganic drugs or organic drugs of the local and systemic type that act on the nervous system, hypnotics, sedatives, narcotic antagonists, psychic energizers, tranquilizers, muscle relaxants, antiparkinson agents, analgesics, antipyretics, anti-inflammatory, anesthetics, antispasmodics, antiulcer, prostaglandins, anti-microbials, anti-malarials, antivirals, hormones, androgenic steroids, estrogenic steroids, progestational steroids, corticosteroids, sympathominetic amines, cardiovascular drugs, diuretics, neoplastics, hypoglycemic, nutritional agents, vitamins, amino acids, essential elements, ophthalmic drugs, and the like. Various drugs and exemplified doses are further described in The Goodman's and Gilman's Pharmacological Basis of Therapeutics, edited by Brunton, L. L., 11th Edition, 2006, published by The McGraw-Hill Companies.

The biologically active molecules can be in various forms, such as uncharged molecules, components of molecular complexes, salts, esters, ethers and amides which have solubility characteristics compatible with the polymer. Also, a bioactive molecule that has limited solubility, or is water insoluble can be used in a form that is a water soluble derivative thereof to effectively serve as a solute, and on its release from the polymer, it is converted by the environment including enzymes, hydrolyzed by body pH, or metabolic processes to the original form or to an active form. The bioactive molecule-bearing polymers can have various forms known in the art such as solution, dispersion, paste, cream, particles (e.g., nanoparticles or microparticles), granules, emulsions, suspension, powders, micronized powders, and the like.

As used in this specification, including any appended claims, the singular “a”, “an”, and “the” include plural reference unless the contact dictates otherwise. Thus, for example, a reference to “a polymer” includes more than one such polymer. A reference to “a biologically active molecule” (or “bioactive” molecule) includes more than one such molecule. A reference to “a cell” includes more than one such cell.

The terms “comprising”, “consisting of” and “consisting essentially of” are defined according to their standard meaning. The terms may be substituted for one another throughout the instant application in order to attach the specific meaning associated with each term.

“Alkyl,” “alkoxy,” etc. denote both straight and branched groups; but reference to an individual radical such as “propyl” embraces only the straight chain radical, a branched chain isomer such as “isopropyl” being specifically referred to. “Aryl” denotes a phenyl radical or an ortho-fused bicyclic carbocyclic radical having about nine to ten ring atoms in which at least one ring is aromatic. “Heteroaryl” encompasses a radical attached via a ring carbon of a monocyclic aromatic ring containing five or six ring atoms consisting of carbon and one to four heteroatoms each selected from the group consisting of non-peroxide oxygen, sulfur, and N(Rₓ) wherein Rₓ is absent or is hydrogen, oxo, alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto. “Heteroalkyl” encompasses the replacement of a carbon atom within an alkyl chain with a heteroatom; e.g., replacement with an element other than carbon such as N, S, or O, including both an alkyl interrupted by a heteroatom as well as an alkyl substituted by a heteroatom.

Specific and preferred values listed below for radicals, substituents, and ranges, are for illustration only; they do not exclude other defined values or other values within defined ranges for the radicals and substituents.

Specifically, “alkyl” can include methyl, ethyl, propyl, isopropyl, butyl, iso-butyl, sec-butyl, pentyl, 3-pentyl, hexyl, heptyl, octyl, nonyl, decyl, undecyl, dodecyl, tridecyl, tetradecyl or pentadecyl.

“Alkenyl” can include vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl; 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, 11-dodecenyl, 1-tridecenyl, 2-tridecenyl, 3-tridecenyl, 4-tridecenyl, 5-tridecenyl, 6-tridecenyl, 7-tridecenyl, 8-tridecenyl, 9-tridecenyl, 10-tridecenyl, 11-tridecenyl, 12-tridecenyl, 1-tetradecenyl, 2-tetradecenyl, 3-tetradecenyl, 4-tetradecenyl, 5-tetradecenyl, 6-tetradecenyl, 7-tetradecenyl, 8-tetradecenyl, 9-tetradecenyl, 10-tetradecenyl, 11-tetradecenyl, 12-tetradecenyl, 13-tetradeceny, 1-pentadecenyl, 2-pentadecenyl, 3-pentadecenyl, 4-pentadecenyl, 5-pentadecenyl, 6-pentadecenyl, 7-pentadecenyl, 8-pentadecenyl, 9-pentadecenyl, 10-pentadecenyl, 11-pentadecenyl, 12-pentadecenyl, 13-pentadecenyl, 14-pentadecenyl.

“Alkoxy” can include methoxy, ethoxy, propoxy, isopropoxy, butoxy, iso-butoxy, sec-butoxy, pentoxy, 3-pentoxy, hexoxy, heptyloxy, octyloxy, nonyloxy, decyloxy, undecyloxy, dodecyloxy, tridecyloxy, tetradecyloxy, or pentadecyloxy; “alkanoyl” can include acetyl, propanoyl, butanoyl, pentanoyl, hexanoyl, heptanoyl, octanoyl, nonanoyl, decanoyl, undecanoyl, dodecanoyl, tridecanoyl, tetradecanoyl, or pentadecanoyl; “cycloalkyl” can include cyclopropyl, cyclobutyl, cyclopentyl, cyclohexyl, cycloheptyl, or cyclooctyl.

“Aryl” can include phenyl, indenyl, 5,6,7,8-tetrahydronaphthyl, or naphthyl. “Heteroaryl” can include furyl, imidazolyl, tetrazolyl, pyridyl, (or its N-oxide), thienyl, pyrimidinyl (or its N-oxide), indolyl, or quinolyl (or its N-oxide).

The terms “patient”, “recipient”, and “subject”, are used interchangeably and, for the purposes of the present invention, include both prokaryotic and eukaryotic cells, such as human cells and non-human animal cells (e.g., mammal cells). Polymers of the subject invention may be administered to such cells in vitro or in vivo. Thus, the methods of administration are applicable to both human therapy and veterinary applications, as well as research applications in vitro or within animal models.

As used herein, an “effective amount” of polymer or bioactive molecule is that amount effective to bring about the physiological changed desired in the cell, tissue, organ, organ system, etc. to which the polymers are administered. The term “therapeutically effective amount” means the amount of a compound or composition that, when administered to a patient, is sufficient to effect the desired therapy. Thus, in the context of the invention, the term “therapeutically effective amount” means that amount of polymers (or bioactive agent(s) bonded thereto), alone or in combination with another agent according to the particular aspect of the invention, that elicits the biological or medicinal response in a cell, tissue, organ, or organ system that is being sought by a researcher, veterinarian, medical doctor or other clinician, which can include alleviation of one or more of the symptoms of the disease or disorder being treated. The therapeutically effective amount will vary depending on the compound, the disease and its severity and the age, weight, etc., of the patient to be treated.

For example, if the bioactive molecule is a therapeutic agent, an effective amount of the polymeric material (polymer) bearing the bioactive molecule is that amount sufficient to treat a pathological condition (e.g., a disease or other disorder) in the cells in vitro or in vivo to which the polymers are administered. Thus, in the case of cancer, and in which the bioactive molecule is an anti-cancer agent, the therapeutically effective amount of the bioactive molecule may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the bioactive molecule may prevent growth and/or kill existing cancer cells, it may be cytostatic and/or cytotoxic. For cancer therapy, efficacy can, for example, be measured by assessing the time to disease progression (TTP) and/or determining the response rate (RR).

As used herein, the term “growth inhibitory amount” of the bioactive molecule or polymer bearing a bioactive molecule, refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

“Treating” or “treatment” of any disease or disorder refers to one or more of the following: (1) ameliorating the disease or disorder (i.e., arresting or reducing the development of the disease or at least one of the clinical symptoms thereof); (2) ameliorating at least one physical parameter, which may not be discernible by the patient; (3) inhibiting the disease or disorder, either physically, (e.g., stabilization of a discernible symptom), physiologically, (e.g., stabilization of a physical parameter), or both; and (4) delaying the onset of the disease or disorder.

The term “pharmaceutically acceptable salt” refers to a salt of a parent compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

The term “pharmaceutically acceptable carrier” refers to a diluent, adjuvant, excipient or vehicle with which a polymer of the invention can be administered.

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The term “preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease), or delay of onset of the disease or disorder.

The term “prodrug” refers to a derivative of a drug molecule that requires a transformation within the body to release or otherwise provide the active drug. Prodrugs are frequently, although not necessarily, pharmacologically inactive until converted to the parent drug. A hydroxyl containing drug may be converted to, for example, an ester, carbonate, acyloxyalkyl or a sulfonate prodrug, which may be hydrolyzed in viva to provide the hydroxyl compound. Prodrugs for drugs with functional groups different than those listed above are well known to the skilled artisan.

The term “promoiety” refers to a form of protecting group that when used to mask a functional group within a drug molecule converts the drug into a prodrug. Typically, the promoiety will be attached to the drug via a bond or bonds that are cleaved by enzymatic or non-enzymatic mechanisms in vivo.

The term “protecting group” refers to a grouping of atoms that, when attached to a reactive functional group in a molecule, masks, reduces, or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1 8 (John Wiley and Sons, 1971 1996). Representative amino protecting groups include, but are not limited to, formyl, acetyl, trifluoroacetyl, benzyl, benzyloxycarbonyl (“CBz”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

As used herein, a “biomaterial” may be defined as a material that is substantially insoluble in body fluids and tissues and that is designed and constructed to be placed in or onto the body or to contact fluid or tissue of the body. Ideally, a biomaterial will not induce undesirable reactions in the body such as blood clotting, tissue death, tumor formation, allergic reaction, foreign body reaction (rejection) or inflammatory reaction; will have the physical properties such as strength, elasticity, permeability and flexibility required to function for the intended purpose; can be purified, fabricated and sterilized easily; and will substantially maintain its physical properties and function during the time that it remains implanted in or in contact with the body. A “biostable” material is one that is not broken down by the body, whereas a “biocompatible” material is one that is not rejected by the body.

As used herein, a “medical device” may be defined as a device that has surfaces that contact blood or other bodily tissues in the course of their operation. This can include, for example, extracorporeal devices for use in surgery such as blood oxygenators, blood pumps, blood sensors, tubing used to carry blood and the like which contact blood which is then returned to the patient. This can also include implantable devices such as vascular grafts, stents, electrical stimulation leads, heart valves, orthopedic devices, catheters, shunts, sensors, replacement devices for nucleus pulposus, cochlear or middle ear implants, intraocular lenses, and the like.

### Materials and Methods

Polymer Synthesis. Monomer was pipetted into a dry 100 mL Schlenk tube equipped with a stir bar and glass stopcock and dried by heating the vessel in an oil bath at 45° C. under full vacuum (10⁻³ mmHg) for 24 h. After 24 h the reaction vessel was backfilled with Argon and first-generation Grubbs' Ru catalyst (100:1/monomer:catalyst) was added. The full vacuum was placed back on the polymerization reaction after ½ h. Additional catalyst was added 60 h into the polymerization to ensure maximum possible couplings. Upon completion of 120 h of reaction time the polymerization was quenched in THF or CHCl₃ with a few drops of ethyl vinyl ether added into the solvent. The ruthenium catalyst is removed via complexation by extracting the organic layer with tris(hydroxymethyl)phosphine (THP) 1M solution. The resultant organic layer is washed concentrated NaHCO₃ (1×30 mL) and brine (1×30 mL) and dried over MgSO₄ followed by rotary evaporation to yield the pure polymer.

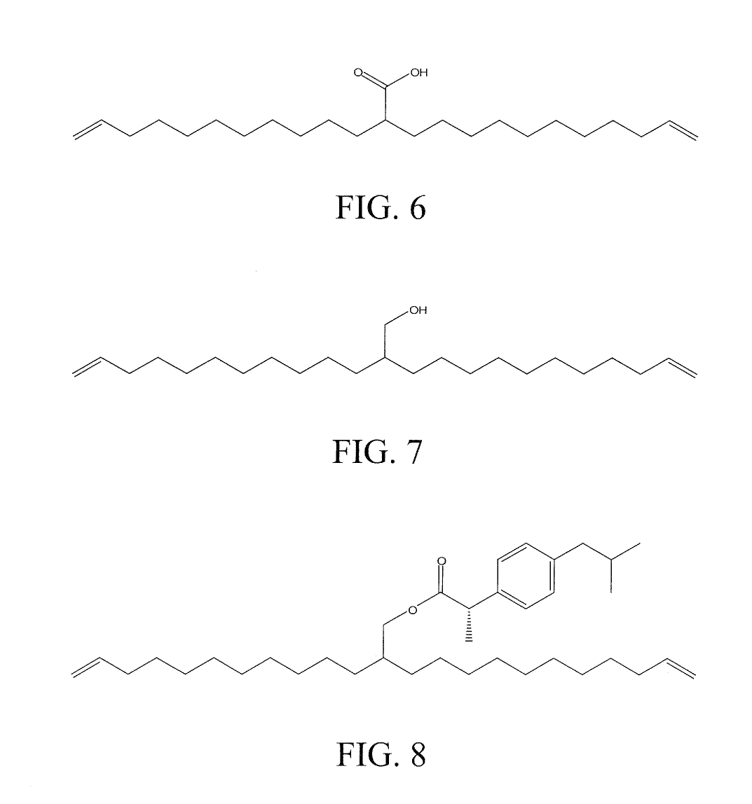

2-(undec-10-enyl)tridec-12-enoic acid. To a 500 mL three neck round-bottom flask equipped with a stir bar, and addition funnel was added diethyl malonate (7.16 g, 0.045 mol), 11-bromoundec-1-ene (23.0 g, 0.099 mol), and 150 mL THF. NaH (3.22 g, 0.134 mol) was added through a powder funnel over 10 minutes and then allowed to stir at room temperature for 1 h and at reflux for an additional day. The reaction was monitored by TLC using a 3:1 hexanes:ethyl acetate mobile phase. More NaH or alkenyl bromide is added if monoalkylated product exists. The mixture was cooled to room temperature and water was added slowly to neutralize the remaining NaH. Then 100 mL of water (total ˜125 mL) and ethanol (100 mL) were added along with NaOH (15 g, 0.375 mol) and the reaction was refluxed for 24 h. The reaction was monitored for the disappearance of the diester using TLC (3:1 hexanes:ethyl acetate mobile phase). The solution was neutralized with concentrated HCl and extracted with diethyl ether. Following the evaporation of the diethyl ether, Decalin (40 mL), and a catalytic amount of DMAP were added to a 250 mL round-bottom flask equipped with a stir bar and a condenser. The flask was lowered into a 190° C. oil bath and decarboxylation was observed by excessive frothing caused by the decarboxylation. Once the frothing ceased the reaction was stirred for an additional 2 h and the reaction was allowed to cool to room temperature. The crude mixture was flashed through a plug of silica gel using hexane as the eluent to remove the Decalin—the Decalin could be observed as a clear ring moving through the plug. Once the Decalin was removed, the eluent was switched to ethyl acetate to remove the desired product (shown in FIG. 6). ¹H NMR (300 MHz, CDCl₃): δ 1.22-1.55 (br, 28H), 1.63 (m, 4H), 2.05 (q, 4H), 2.06 (q, 4H), 2.37 (m, 1H), 4.98 (m, 4H), 5.82 (m, 2H). ¹³C NMR (75 MHz, CDCl₃): δ 27.58, 29.15, 29.34, 29.68, 29.75, 29.77, 32.37, 34.03, 45.75, 114.31, 139.44, 183.14.

2-(undec-10-enyl)tridec-12-en-1-ol. To a 250 mL three neck round-bottom flask equipped with a stir bar was added 125 mL of dry THF upon which LiAlH₄ powder (4.6 g 0.109 mol) was stirred in slowly. To this slurry was added impure 9,9 acid in the form of a colorless oil (10 g, 0.027 mol) over 15 minutes via syringe. The reaction was placed under Ar and left to stir at room temperature for 18 hours. The reaction was quenched with water and acidified with concentrated HCl. A small amount of diethyl ether was added and the solution was extracted with 1M HCl (2×50 mL) and brine (2×50 mL). The organic layer was dried over MgSO₄ and concentrated down to a light yellow oil. This product was purified by flash chromatography using 9:1 (hexane:ethyl acetate) mobile phase yielding 4.1 g of the desired primary alcohol (shown in FIG. 7). ¹H NMR (300 MHz, CDCl₃): δ 1.3 (m, br, 33H), 1.91 (s, 1H), 2.03 (q, 4H), 3.54 (d, 2H), 4.96 (m, 4H), 5.81 (m, 2H). ¹³C NMR (75 MHz, CDCl₃): δ 15.25, 26.94, 28.99, 29.20, 29.57, 29.67, 30.14, 30.98, 33.88, 40.57, 65.61, 114.13, 139.21.27.58, 29.15, 29.34, 29.68, 29.75, 29.77, 32.37, 34.03, 45.75, 114.31, 139.44, 183.14.

### EXAMPLES

### Example 1

### Coupling of a 9,9 Primary Alcohol to Either the Ibuprofen or Naproxen Drug Molecules

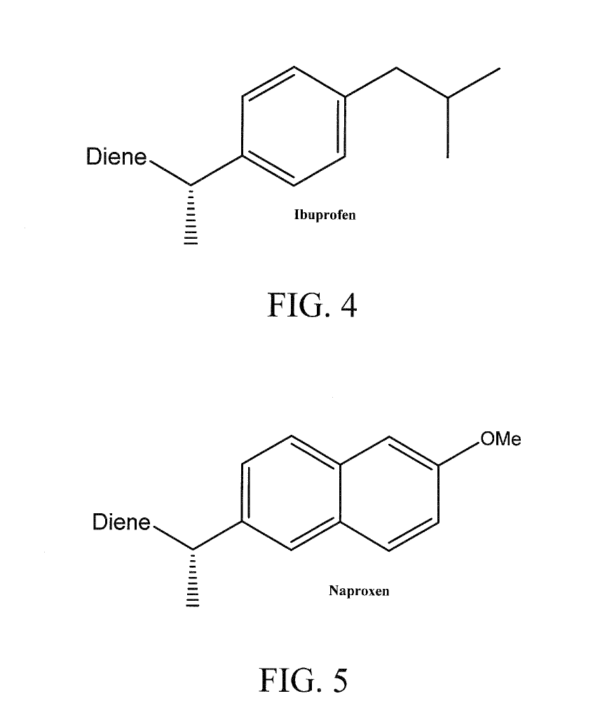

To a flame dried three neck round-bottom flask equipped with stir bar and under Ar was added the 9,9 alcohol (1.25 equiv to drug). To this oil was added 150 mL of dry THF followed by the addition of either the Ibuprofen or Naproxen drug molecule (the chemical structures of which are shown in FIGS. 4 and 5, respectively), DMAP (1.25 equiv to drug) and EDCI (1.25 equiv to drug) upon which the reaction turned cloudy. The reaction was sealed and kept stirring at RT for 24 h until the reaction mixture went clear and grayish sticky salts crashed out of solution. After monitoring completion by TLC, 100 mL of water was added to dissolve the salts along with 50 mL of diethyl ether to separate organic from aqueous. The organic layer was washed with water (2×100 mL) and brine (2×100 mL) and then dried over MgSO₄. The resultant monomers were purified via column chromatography to give colorless oils in 78% yields.

(S)-2-(undec-10-enyl)tridec-12-enyl 2-(4-isobutylphenyl)propanoate. The pure product (shown in FIG. 8) was obtained in % yield after purification using flash chromatography using 14:1 (hexane:ethyl acetate) mobile phase. ¹H NMR (300 MHz, CDCl₃): δ 0.88 (d, 6H), 1.14-1.40 (m, b, 32H), 1.47 (d, 3H), 1.54 (b, 1H), 1.83 (m, 1H), 2.03 (q, 4H), 2.42 (d, 2H), 3.67 (q, 1H), 3.94 (o, 2H), 4.93 (m, 4H), 5.80 (m, 2H), 7.06 (d, 2H), 7.18 (d, 2H). ¹³C NMR (75 MHz, CDCl₃): δ 18.44, 22.59, 26.84, 26.87, 29.18, 29.38, 29.73, 29.79, 29.81, 30.14, 30.39, 31.34, 31.44, 34.03, 37.53, 45.28, 45.52, 67.44, 114.31, 127.40, 129.42, 138.13, 139.41, 140.57, 175.03. EI/HRMS [M+1]: calcd for C₃₇H₆₂O₂, 539.4823 g/mol. found, 539.4809 g/mol.

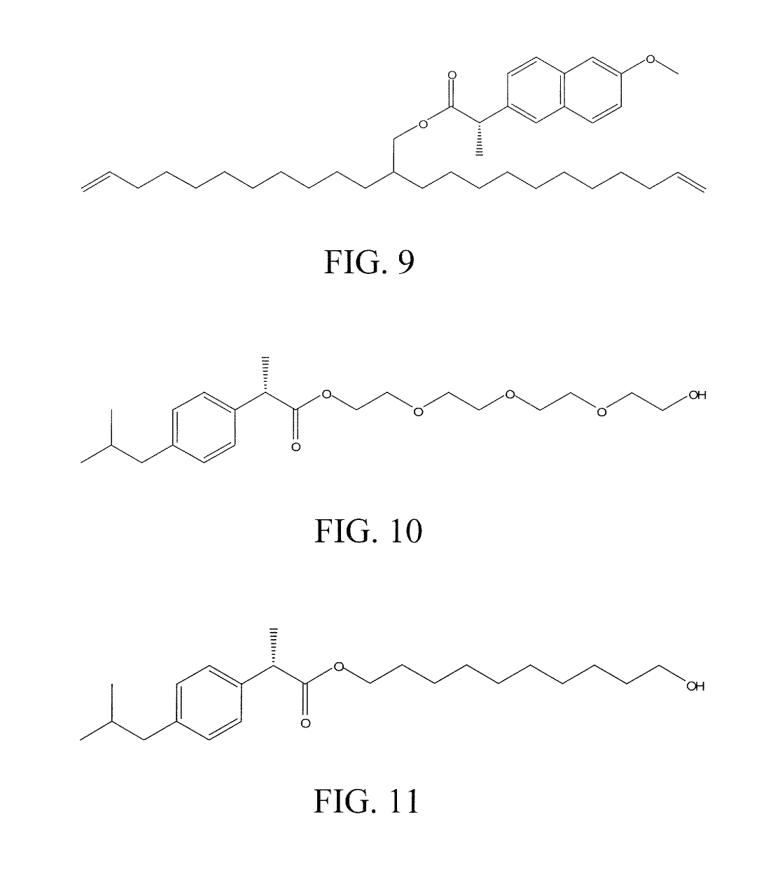

(S)-2-(undec-10-enyl)tridec-12-enyl 2-(6-methoxynapthalen-2-yl)propanoate. The pure product (shown in FIG. 9) was obtained in % yield after purification using flash chromatography using 14:1 (hexane:ethyl acetate) mobile phase. ¹H NMR (300 MHz, CDCl₃): δ 1.17-1.40 (br, 33H), 1.57 (d, 3H), 2.02 (m, 4H), 3.83 (q, 1H), 3.89 (s, 3H), 3.97 (m, 2H), 4.94 (m, 4H), 5.80 (m, 2H), 7.08-7.13 (m, 2H), 7.39 (m, 1H), 7.66 (m, 3H). ¹³C NMR (75 MHz, CDCl₃): δ 14.39, 18.45, 26.81, 26.87, 29.15, 29.35, 29.68, 29.73, 29.77, 30.09, 31.38, 31.47, 34.02, 37.52, 45.82, 55.43, 60.55, 67.62, 105.76, 114.30, 119.09, 126.12, 126.50, 127.21, 129.15, 129.43, 133.88, 136.04, 139.40, 157.80, 174.92. EI/HRMS [M+1]: calcd for C₃₈H₅₈O₃, 563.4459 g/mol. found, 563.4466 g/mol.

### Example 2

### Coupling of Ibuprofen or Naproxen to Either Decanediol or Tetraethylene Glycol

To a flame dried 250 mL three neck round-bottom flask with a stir bar was added the drug (2 g of either Ibuprofen or Naproxen). This material was dissolved in 100 mL of dry chloroform and then the carbonyl diimidazole (1.1 equiv to drug) was slowly added over 5 minutes upon which lots of CO₂ bubbles evolved for approximately 10 minutes. The solution was allowed to stir for 2 h at RT to ensure complete formation of the activated acid. An excess of diol (4 equiv to drug of either decanediol or tetraethylene glycol) was added quickly and allowed to stir at room temperature for 48 h. Upon completion of the reaction, 25 mL of chloroform was added and this organic layer was washed with water (2×75 mL) and brine (2×75 mL) and was dried over MgSO₄. The organic layer was then concentrated to a semi-viscous oil and was purified by flash chromatography using 1:1 (ethyl acetate:diethyl ether) for the tetraethylene glycol esters and 1:1 (hexane:THF) for the decanediol esters.

(S)-2-(2-(2-(2-hydroxyethoxy)ethoxy)ethoxy)ethyl 2-(4-isobutylphenyl)propanoate. The pure product (shown in FIG. 10) was obtained in % yield after purification using flash chromatography using 14:1 (hexane:ethyl acetate) mobile phase. ¹H NMR (300 MHz, CDCl₃): δ 0.90 (d, 6H), 1.49 (d, 3H), 1.84 (m, 1H), 2.43 (d, 2H), 2.66 (s, 1H), 3.50-3.80 (m, br, 15H), 4.22 (m, 2H), 7.08 (d, 2H), 7.20 (d, 2H). ¹³C NMR (75 MHz, CDCl₃): δ 0.14, 18.70, 22.53, 30.32, 45.14, 45.16, 61.86, 64.01, 69.22, 70.49, 70.68, 70.69, 70.77, 72.65, 127.34, 129.42, 137.82, 140.62, 174.84. EI/HRMS [M+1]: calcd for C₂₁H₃₄O₆, 383.2482 g/mol. found, 383.2481 g/mol.

(S)-10-hydroxydecyl 2-(4-isobutylphenyl)propanoate. The pure product (shown in FIG. 11) was obtained in % yield after purification using flash chromatography using 14:1 (hexane:ethyl acetate) mobile phase. ¹H NMR (300 MHz, CDCl₃): δ 0.87 (d, 6H), 1.20-1.1.30 (br, 12H), 1.46 (d, 3H), 1.54 (m, 4H), 1.82 (m, 2H), 2.02 (s, 1H), 2.42 (d, 2H), 3.60 (t, 2H), 3.65 (q, 1H), 4.03 (t, 2H), 7.06 (d, 2H), 7.18 (d, 2H). ¹³C NMR (75 MHz, CDCl₃): δ 18.59, 22.52, 25.89, 28.66, 29.27, 29.54, 29.62, 30.31, 32.91, 45.19, 45.36, 63.08, 64.90, 127.29, 129.39, 138.05, 140.54, 174.99.

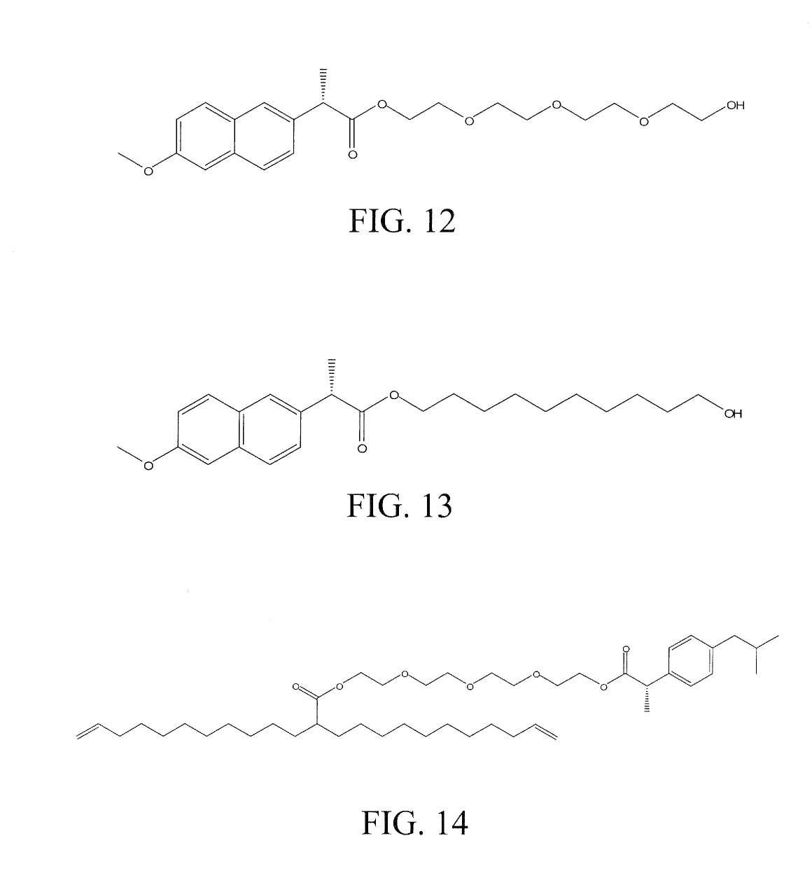

(S)-2-(2-(2-(2-hydroxyethoxy)ethoxy)ethoxy)ethyl 2-(6-methoxynaphthalen-2-yl)propanoate. The pure product (shown in FIG. 12) was obtained in % yield after purification using flash chromatography using 14:1 (hexane:ethyl acetate) mobile phase. ¹H NMR (300 MHz, CDCl₃): δ 1.57 (d, 3H), 2.67 (s,br, 1H), 3.48-3.88 (m, br, 14H), 3.88 (q, 1H), 3.89 (s, 3H), 4.23 (t, 2H), 7.11 (d, 1H), 7.15 (d, 1H), 7.40 (d, 1H), 7.43 (d, 1H), 7.67 (s, 1H), 7.71 (s, 1H) ¹³C NMR (75 MHz, CDCl₃): δ 0.12, 18.64, 45.45, 55.40, 61.81, 64.08, 69.14, 70.40, 70.56, 70.60, 70.66, 72.59, 105.67, 119.07, 126.10, 126.40, 127.22, 129.03, 129.38, 133.80, 135.75, 157.74, 174.73. EI/HRMS [M+1]: calcd for C₂₂H₃₀O₇, 407.2064 g/mol. found, 407.2044 g/mol.

(S)-10-hydroxydecyl 2-(6-methoxynaphthalen-2-yl)propanoate. The pure product (shown in FIG. 13) was obtained in % yield after purification using flash chromatography using 14:1 (hexane:ethyl acetate) mobile phase. ¹H NMR (300 MHz, CDCl₃): δ 1.12-1.30 (br, 12H), 1.42 (s, 1H), 1.52 (m, 4H), 1.55 (d, 3H), 3.61 (t, 2H), 3.82 (q, 1H), 3.89 (s, 3H), 4.04 (t, 2H), 7.1 (s, 1H), 7.13 (d, 1H), 7.38 (d, 1H), 7.65 (s, 1H), 7.66 (s, 1H), 7.69 (s, 1H) ¹³C NMR (75 MHz, CDCl₃): δ 18.70, 25.91, 25.98, 28.75, 29.33, 29.57, 29.64, 33.01, 45.76, 55.52, 63.27, 65.08, 105.84, 119.11, 126.12, 126.50, 127.27, 129.16, 129.48, 133.88, 136.09.

### Example 3

### Coupling of the 9,9 Acid Diene to Either Decanediol Ester Drugs or Tetraethylene Glycol Ester Drugs

To a flame dried three neck round-bottom flask equipped with stir bar and placed under Ar was charged the 9,9 acid diene. To this solid was added 150 mL of dry THF followed by the addition of either the decanediol ester drug or the tetraethylene glycol ester drug (1.25 equiv to the 9,9 acid diene), DMAP (1.25 equiv to the 9,9 acid diene) and EDCI (1.25 equiv to the 9,9 acid diene) upon which the reaction turned cloudy. The reaction was sealed and kept stirring at RT for 24 h until the reaction mixture went clear and grayish sticky salts crashed out of solution. After monitoring completion by TLC, 100 mL of water was added to dissolve the salts along with 50 mL of diethyl ether to separate organic from aqueous. The organic layer was washed with water (2×100 mL) and brine (2×100 mL) and then dried over MgSO₄. The resultant monomers were all purified via column chromatography to give colorless oils in 75% to 90% yields.

(S)-14-(4-isobutylphenyl)-13-oxo-3,6,9,12-tetroxapentadecyl 2-(undec-10-enyl)tridec-12-enoate. The pure product (shown in FIG. 14) was obtained in % yield after purification using flash chromatography using 14:1 (hexane:ethyl acetate) mobile phase. ¹H NMR (300 MHz, CDCl₃): δ 0.87 (d, 6H), 1.20-1.60 (br, m, 34H), 1.82 (m, 1H), 1.98 (q, 4H), 2.31 (br, 1H), 2.41 (d, 2H), 3.50-3.75 (br, m, 13H), 4.19 (m, 4H), 4.91 (m, 4H), 5.78 (m, 2H), 7.05 (d, 2H), 7.17 (d, 2H). ¹³C NMR (75 MHz, CDCl₃): δ 18.75, 22.57, 27.58, 29.12, 29.31, 29.65, 29.73, 29.75, 30.34, 32.59, 33.98, 45.22, 45.81, 63.22, 64.03, 69.27, 69.48, 70.73, 70.76, 70.83, 114.29, 127.37, 129.45, 137.87, 139.37, 140.65, 174.82, 176.65. EI/HRMS [M+1]: calcd for C₄₅H₇₆O₇, 729.5664 g/mol. found, 729.5689 g/mol.

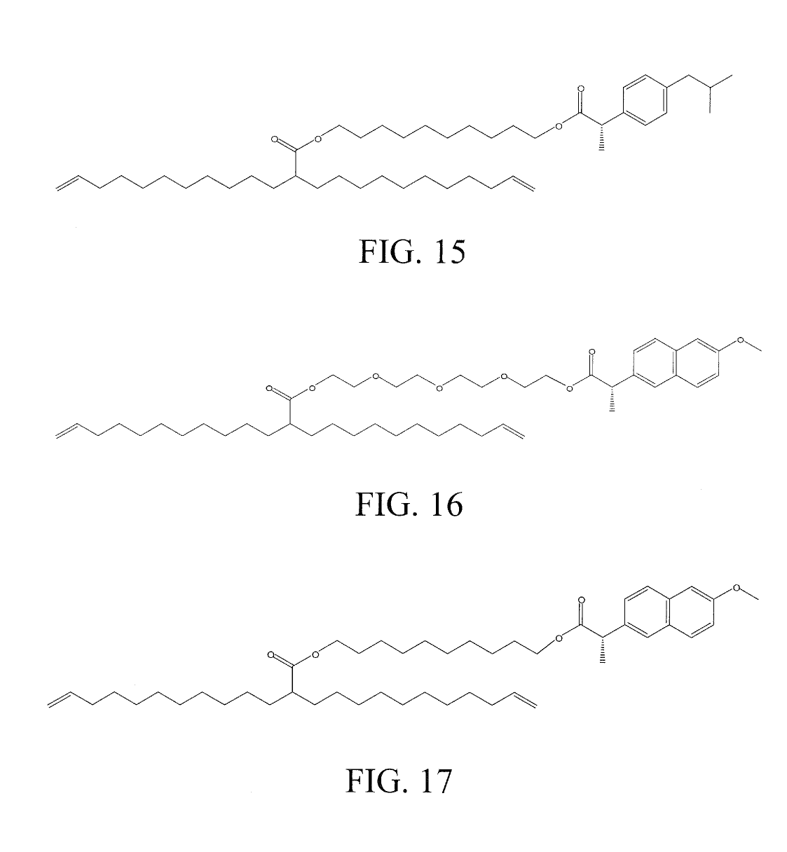

(S)-10-(2-(4-isobutylphenyl)propanoyloxy)decyl 2-(undec-10-enyl)tridec-12-enoate. The pure product (shown in FIG. 15) was obtained in % yield after purification using flash chromatography using 14:1 (hexane:ethyl acetate) mobile phase. ¹H NMR (300 MHz, CDCl₃): δ 0.87 (d, 6H), 1.20-1.65 (br, m, 51H), 1.82 (m, 1H), 2.01 (q, 4H), 2.28 (m, 1H), 2.42 (d, 2H), 3.65 (q, 1H), 4.04 (q, 4H), 4.92 (m, 4H), 5.78 (m, 2H), 7.06 (d, 2H), 7.18 (d, 2H). ¹³C NMR (75 MHz, CDCl₃): δ 18.66, 22.57, 25.97, 26.18, 27.67, 28.75, 28.94, 29.14, 29.33, 29.36, 29.43, 29.67, 29.72, 29.76, 30.37, 32.75, 34.00, 45.25, 45.41, 46.05, 64.29, 64.91, 114.30, 127.34, 129.44, 138.12, 139.38, 140.58, 174.96, 176.82. EI/HRMS [M+1]: calcd for C₄₇H₈₀O₄, 709.6129 g/mol. found, 709.6111 g/mol.

(S)-14-(6-methoxynaphthalen-2-yl)-13-oxo-3,6,9,12-tetroxapentadecyl 2-(undec-10-enyl)tridec-12-enoate. The pure product (shown in FIG. 16) was obtained in % yield after purification using flash chromatography using 14:1 (hexane:ethyl acetate) mobile phase. ¹H NMR (300 MHz, CDCl₃): δ 1.20-1.45 (br, m, 32H), 1.55 (d, 314), 2.00 (q, 4H), 2.32 (m, 1H), 3.45-3.65 (m, br, 12H), 4.20 (q, 1H), 4.21 (s, 3H), 4.22 (m, 4H), 4.92 (m, 4H), 5.76 (m, 2H), 7.05-7.15 (m, 2H), 7.38 (d, 1H), 7.67 (d, 3H). ¹³C NMR (75 MHz, CDCl₃): δ 18.70, 27.55, 29.09, 29.28, 29.62, 29.69, 29.72, 32.56, 33.95, 45.50, 45.78, 55.43, 63.19, 64.09, 69.20, 69.42, 70.65, 70.69, 70.71, 105.74, 114.27, 119.10, 126.13, 126.41, 127.25, 129.08, 129.41, 133.85, 135.78, 139.33, 157.81, 174.70, 176.61. EI/HRMS [M+1]: calcd for C₄₆H₇₂O₈, 753.5300 g/mol. found, 753.5305 g/mol.

(S)-10-(2-(6-methoxynaphthalen-2-yl)propanoyloxy)decyl 2-undec-10-enyl)tridec-12-enoate. The pure product (shown in FIG. 17) was obtained in % yield after purification using flash chromatography using 14:1 (hexane:ethyl acetate) mobile phase. ¹H NMR (300 MHz, CDCl₃): δ 1.15-1.65 (br, m, 50H), 1.99 (q, 4H), 2.29 (m, 1H), 3.82 (q, 1H), 3.89 (s, 3H), 4.04 (t, 4H), 4.94 (m, 4H), 5.79 (m, 2H), 7.05-7.15 (m, 2H), 7.39 (d, 1H), 7.66 (d, 3H) ¹³C NMR (75 MHz, CDCl₃): δ 18.69, 25.98, 26.14, 27.66, 28.74, 28.92, 29.13, 29.32, 29.40, 29.60, 29.66, 29.71, 29.75, 32.74, 33.99, 45.72, 46.04, 55.45, 64.29, 65.03, 105.78, 114.29, 119.10, 126.09, 126.45, 127.24, 129.14, 129.44, 133.86, 136.05, 139.38, 157.81, 174.87, 176.83._EI/HRMS [M+1]: calcd for C₄₈H₇₆O₅, 733.5766 g/mol. found, 733.5768 g/mol.

All patents, patent applications, provisional applications, and publications referred to or cited herein, supra or infra, are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

We claim:

1. A polymer comprising a plurality of repeating diene monomers having coupled thereto at least one biologically active molecule through at least one non-amide linking moiety (linker).

2. The polymer of claim 1, wherein the linker is least one moiety selected from the group consisting of an ester, carbonate, carbomate, and ether.

3. The polymer of claim 1, wherein linker comprises an ether moiety and carbamate moiety.

4. The polymer of claim 1, wherein the diene monomer is selected from the group consisting of 3,3 acid, 6,6 acid, 9,9 acid, and 18,18 acid.

5. The polymer of claim 1, wherein the monomer further comprises at least one spacer interposed between the biologically active molecule and the linker.

6. The polymer of claim 5, wherein the spacer is at least one moiety selected from the group consisting of methoxy and glycol.

7. The polymer of claim 6, wherein the spacer is at least one ethylene glycol moiety.

8. The polymer of claim 6, wherein the spacer is diethylene glycol or triethylene glycol.

9. The polymer of claim 1, wherein the spacer is at least one alkyl or diol moiety.

10. The polymer of claim 1, wherein the at least one linker, the at least one spacer, or both are degradable by enzymatic cleavage, chemical hydrolysis, or both.

11. The polymer of claim 1, wherein the biologically active molecule is selected from the group consisting of analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, anti epileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, pesticides, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

12. A pharmaceutical composition comprising a polymer of claim 1; and a pharmaceutically acceptable carrier.

13. A method for making a polymer of claim 1, comprising providing diene monomers coupled to at least one biologically active molecule through at least one non-amide linking moiety (linker); and polymerizing the monomers to produce the polymer.

14. The method of claim 13, wherein said providing comprises coupling the at least one biologically active molecule to the diene through at least one linker.

15. A method for delivering a biologically active molecule to a subject, comprising administering a polymer of claim 1 to the subject.

16. A device comprising a polymer of claim 1.

17. The device of claim 16, wherein the device comprises one or more substrates (surfaces) coated with the polymer or coated with a composition comprising the polymer.

18. The device of claim 16, wherein the device is an implantable or deployable medical device.
